(19) United States
(12) Patent Application Publication
(10) Pub. No.: US 2025/0368872 A1
(43) Pub. Date: Dec. 4, 2025

(54) PROTEIN CROSSLINKING AGENT

(71) Applicants: CANON KABUSHIKI KAISHA, Tokyo (JP); CANON VIRGINIA, INC., Newport News, VA (US)

(72) Inventors: YUMA KOBAYASHI, Kanagawa (JP); KEIGO MIZUSAWA, Tokyo (JP); KEI INOUE, Kanagawa (JP); WAKA HASEGAWA, Tokyo (JP)

(21) Appl. No.: 19/297,395

(22) Filed: Aug. 12, 2025

Related U.S. Application Data
(63) Continuation of application No. PCT/JP2024/004780, filed on Feb. 13, 2024.

(30) Foreign Application Priority Data
Feb. 14, 2023 (JP) 2023-020823
Feb. 1, 2024 (JP) 2024-014496

(51) Int. Cl. C09J 189/00; B32B 7/12; B32B 9/04; C07K 14/435; C08F 8/34; C08J 3/24; C09J 5/00

(52) U.S. Cl. CPC C09J 189/00; B32B 7/12; B32B 9/04; C07K 14/43586; C08F 8/34; C08J 3/246; C09J 5/00; C08F 2810/20; C08J 2389/00; C08J 2429/04

(57) ABSTRACT

A purpose is to provide a protein crosslinking agent which can be easily used. A protein crosslinking agent for a protein having a β-sheet structure is provided, comprising a structural unit derived from one selected from the group consisting of polyethylene, polyvinyl alcohol, polyamine, polyamide, polyester and polyether, and a group containing an aromatic ring, wherein the group containing an aromatic ring has at least one selected from the group consisting of a benzothiazole group, a benzoxazole group, a benzimidazole group and a naphthylazo group, each of which may have a substituent.

### CROSS-REFERENCE TO RELATED APPLICATIONS

This application is a Continuation of International Patent Application No. PCT/JP2024/004780, filed Feb. 13, 2024, which claims the benefit of Japanese Patent Application No. 2023-020823, filed Feb. 14, 2023, and Japanese Patent Application No. 2024-014496, filed Feb. 1, 2024, all of which are hereby incorporated by reference herein in their entirety.

### BACKGROUND

### Field of the Technology

The present disclosure relates to a protein crosslinking agent for a protein having a β-sheet structure, a protein adhesive agent, a protein complex, an article, a compound, a method for crosslinking the protein, and a method for producing the article.

### Description of the Related Art

A protein is a natural macromolecule composed of amino acid sequences. For example, the proteins such as a fibroin, a collagen, and a keratin have excellent mechanical strength and are suitably used as structural materials. In addition, since a protein is highly biocompatible, it is suitably used as a base material of a wearable biosensor device which is attached to a living body and reads biological information such as sweat and pulse, a sealing material, a cell scaffold material, and an in vivo structural material such as an artificial blood vessel or an artificial bone. In addition, proteins have also attracted attention in recent years as substitutes for structural materials such as synthetic plastics because of their low negative impact on the environment.

For example, the properties of protein structural materials can be controlled by various processes. Japanese Patent Laid-open No. 2020-094197 describes a method of making a fibroin solution into a film and describes a method of controlling the mechanical strength by changing the proportion of the β-sheet structure which is the secondary structure of the fibroin molding by using a solvent.

In addition, a method for improving mechanical strength by crosslinking between protein molecules has been studied. Japanese Patent Laid-open No. 2021-147426 describes a method for improving mechanical strength using covalent cross-linking by chemically treating a protein powder under high temperature and high heat.

### SUMMARY

However, when crosslinking is carried out by conventional techniques, there is a problem that the productivity is low because cumbersome chemical operations are required. Therefore, the present disclosure is directed to provide a protein crosslinking agent that can be used simply.

To solve the above problem, the present inventor has made an intense investigation. As a result, it was found that proteins can be easily cross-linked by using a protein crosslinking agent having a group containing an aromatic ring such as a benzothiazole group, since the group containing an aromatic ring adsorbs non-covalently to a protein having a β-sheet structure. The present disclosure has been completed by repeated investigation based on such knowledge.

That is, the present disclosure provides a protein crosslinking agent for a protein having a β-sheet structure, comprising a structural unit derived from one selected from the group consisting of polyethylene, polyvinyl alcohol, polyamine, polyamide, polyester, and polyether, and a group containing an aromatic ring, wherein the group containing an aromatic ring has at least one selected from the group consisting of a benzothiazole group, a benzoxazole group, a benzimidazole group, and a naphthylazo group, each of which may have a substituent.

Features of the present disclosure will become apparent from the following description of embodiments.

### DESCRIPTION OF THE EMBODIMENTS

The present disclosure provides, as an embodiment, a protein crosslinking agent for a protein having a β-sheet structure, comprising a structural unit derived from one selected from the group consisting of polyethylene, polyvinyl alcohol, polyamine, polyamide, polyester, and polyether, and a group containing an aromatic ring, wherein the group containing an aromatic ring has at least one selected from the group consisting of a benzothiazole group, a benzoxazole group, a benzimidazole group, and a naphthylazo group, each of which may have a substituent.

### Protein Crosslinking Agent for Protein Having a β-Sheet Structure

The protein crosslinking agent of the present disclosure for a protein having a β-sheet structure (sometimes referred to simply as the crosslinking agent), comprises a structural unit derived from one selected from the group consisting of polyethylene, polyvinyl alcohol, polyamine, polyamide, polyester, and polyether. Preferably, the structural unit is included in a polymer structure, and the side chain of the polymer structure is bonded to a group containing one or more aromatic rings, and preferably bonded to a group containing a plurality of aromatic rings. The method of bonding a group containing an aromatic ring to a polymer structure is not particularly limited, and the group may be bonded directly or via a linker.

Examples of linkers include structures having a framework of hydrocarbons, oligoethylene glycol, polyethylene glycol, polypropylene glycol, and the like. The bonds used to bond a polymer structure and a group containing an aromatic ring, a polymer structure and a linker, a linker and a group containing an aromatic ring can include, amide bonds, ester bonds, ether bonds, thioether bonds, disulfide bonds, imine bonds, oxime bonds, hydrazide bonds, phosphate ester bonds, and 1,2,3-triazole bonds, but are not limited to.

### Group Containing an Aromatic Ring

The group containing an aromatic ring in this embodiment has at least one of a benzothiazole group, a benzoxazole group, a benzimidazole group, and a naphthylazo group. Specific examples of preferred structures (monovalent groups) are shown in formulae (1) to (18) below. These groups have adsorptivity to proteins having a β structure. However, the group containing an aromatic ring is not limited to the following formulae (1) to (18) as long as it has adsorptivity to a protein having a β structure.

In the following formula, X is either S, O, or NH.

Since S, O, and NH are all divalent, and S, O, and N have close atomic numbers, it is considered that they will adopt similar molecular structures when replaced.

R¹ to R³ independently represent a hydrogen atom, a substituted or unsubstituted aliphatic hydrocarbon group having 1 to 4 carbon atoms, a substituted or unsubstituted aryl or aralkyl group having 6 to 10 carbon atoms, or an alkoxy group. Symbol * indicates a bonding site.

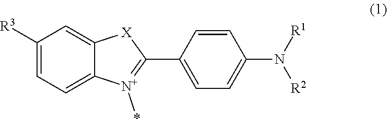

As preferred examples of formula (1), formulae (1-1) to (1-3) can be given.

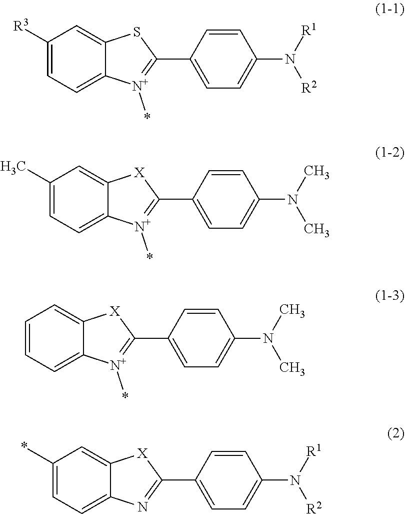

As preferred examples of formula (2), formulae (2-1) to (2-2) can be given.

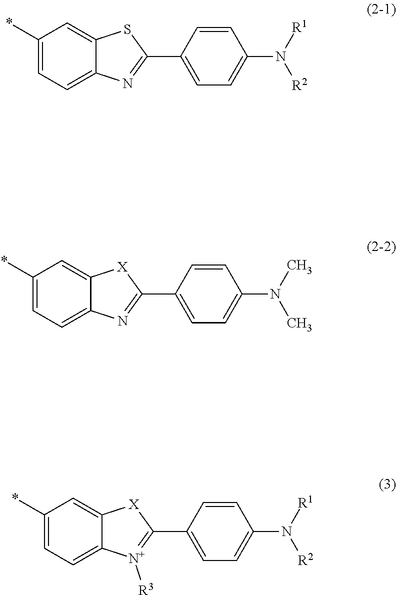

As preferred examples of formula (3), formulae (3-1) to (3-2) can be given.

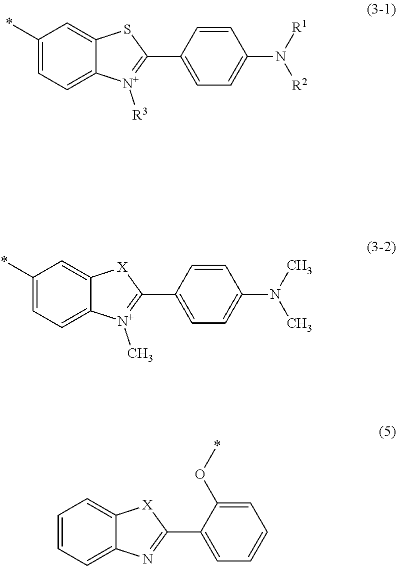

As preferred example of formula (5), formula (5-1) can be given.

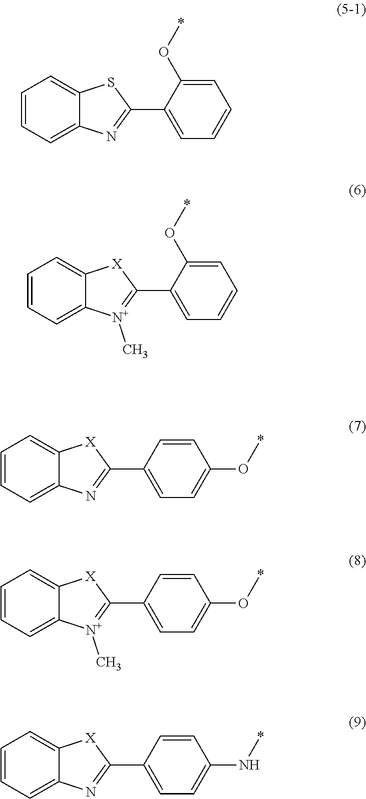

As preferred example of formula (9), formula (9-1) can be given.

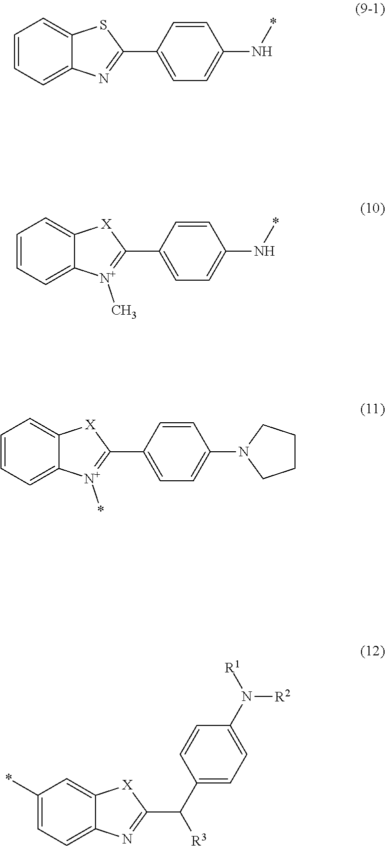

As preferred example of formula (12), formula (12-1) can be given.

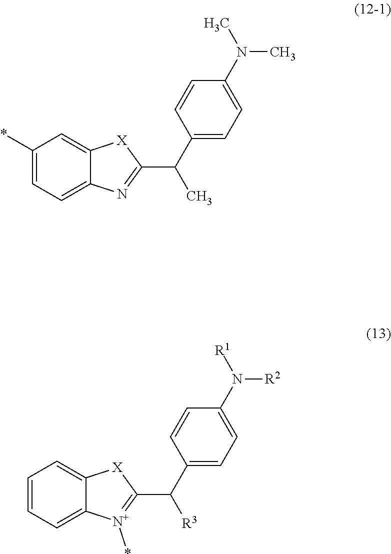

As preferred example of formula (13), formula (13-1) can be given.

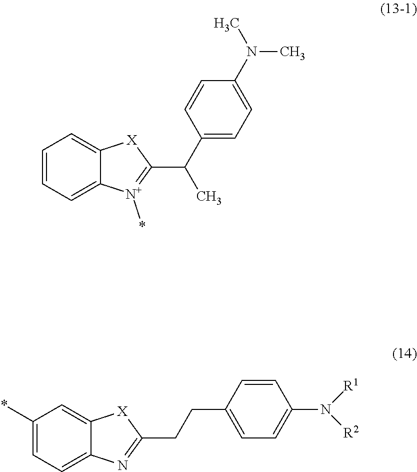

As preferred example of formula (14), formula (14-1) can be given.

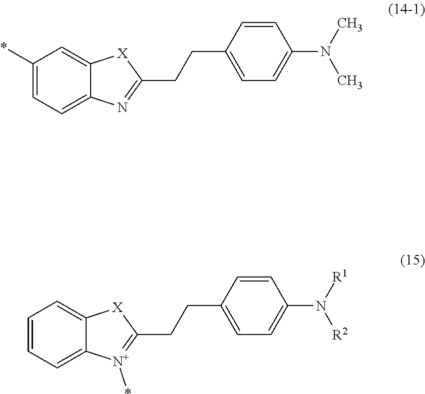

As preferred example of formula (15), formula (15-1) can be given.

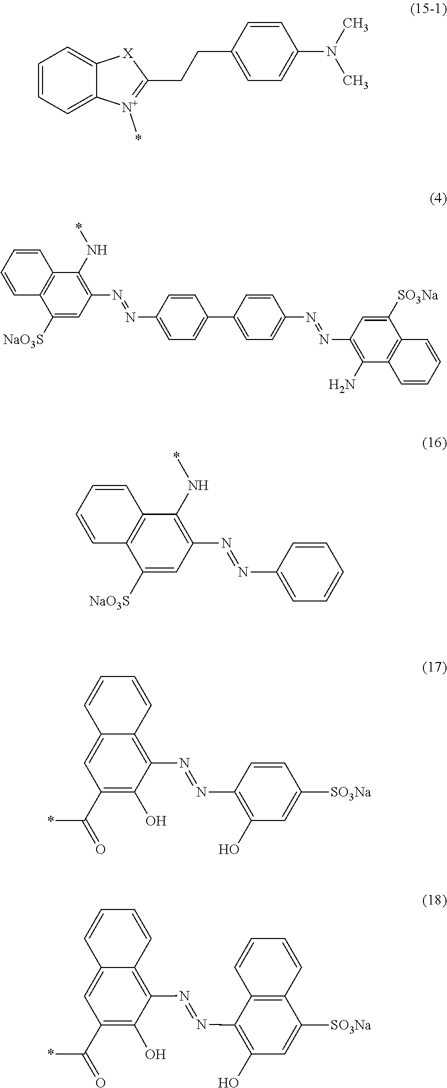

Among them, a monovalent group represented by formula (1-1), formula (2-1), formula (3-1), formula (4), formula (5-1), and formula (9-1) can be given as a group containing a particularly preferable aromatic ring.

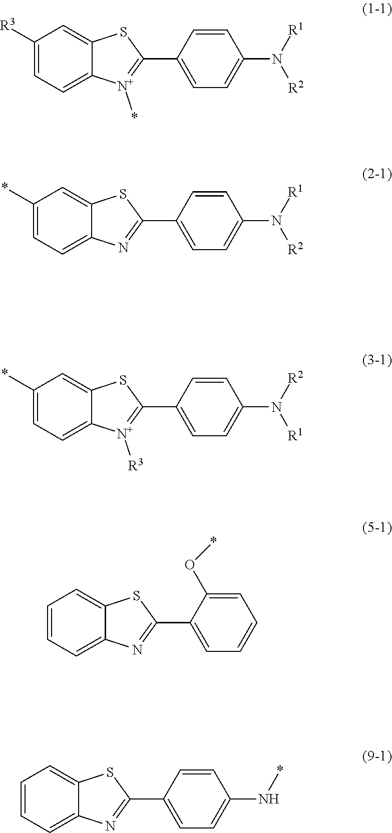

(In the foregoing formulae (1-1), (2-1), (3-1), (5-1), and (9-1), R¹ to R³ independently represent a hydrogen atom, a substituted or unsubstituted aliphatic hydrocarbon group having 1 to 4 carbon atoms, a substituted or unsubstituted aryl or aralkyl group having 6 to 10 carbon atoms, or an alkoxy group, and symbol * indicates a bonding site.)

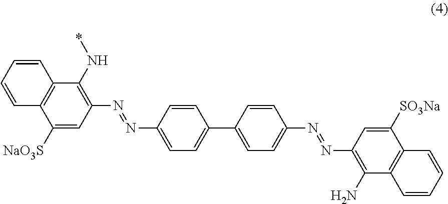

(In the above formula (4), symbol * indicates a bonding site.)

The crosslinking agent has one or more groups containing aromatic rings per molecule, preferably two or more groups containing aromatic rings per molecule. The greater the number of aromatic rings, the more efficient the cross-linking. Although there is no particular upper limit on the number of groups containing aromatic rings, from a synthetic point of view, the number of groups containing aromatic rings per molecule of the crosslinking agent is preferably 100 or less. That is, the number of aromatic rings per molecule of the crosslinking agent is preferably 2 to 100. More preferably, the number of aromatic rings per molecule of the crosslinking agent is 4 to 50.

### Polymer Structure

The crosslinking agent of the present embodiment comprises a structural unit derived from one selected from the group consisting of polyethylene, polyvinyl alcohol, polyamine, polyamide, polyester, and polyether, and preferably comprises a polymer structure comprising these structural units. For example, —CH2—CH2— can be cited as a structural unit derived from polyethylene, and —CH2—CH(OH)— can be cited as an example of a structural unit derived from polyvinyl alcohol.

The polymer structure preferably has a molecular weight of 1000 or more and less than 1 million. If the molecular weight is small, it may not be close enough to the protein to be cross-linked. If the molecular weight is large, sufficient solubility and fluidity may not be ensured and proteins may not be crosslinked. The polymer structure may be a polymer structure consisting of a repetition of one kind of structural unit or a copolymer containing a plurality of kinds of structural units, and when the polymer is a copolymer, it may be an alternate copolymer, a random copolymer, a block copolymer, a graft copolymer, and the like. The polymer structure may contain heteroatoms and may contain nitrogen and silicon. The polymer structure may be derived from a natural polymer, for example, polyamine, cellulose, amylose, starch, collagen, chitin, keratin, natural rubber, or glycoprotein, polypeptide, protein, DNA, RNA, lignin, or asphaltene.

### Crosslinking

The crosslinking agent of the present embodiment can crosslink between proteins having a β-sheet structure.

The crosslinking may be crosslinking between protein molecules. Further, the crosslinking agent may be to crosslink a protein in a solid form or a gel form, and the crosslinking agent may function as an adhesive, for example, a crosslinking agent may be applied to the surface of a protein in a solid form or a gel form, and another protein structure may be brought into close contact therewith, thereby crosslinking between the structures.

### Protein Having a β-Sheet Structure

The protein complex of the present embodiment is a protein complex consisting of a protein having a β-sheet structure. The β-sheet structure is the secondary structure of a protein and refers to a folded structure composed of several polypeptides joined in parallel by hydrogen bonds. However, the state of secondary structure formation depends on the sequence of the protein and the environment around the molecule. For example, a protein having a β-sheet structure may be globular, filamentous, membrane, or glycoprotein, that includes specifically fibroin, streptavidin, immunoglobulin, ovalbumin, ribonuclease, oredoxin, DNA polymerase, glutaminase, ferredoxin, frataxin, green fluorescent protein, lysozyme, amyloid β, and a-synuclein. In addition, a protein having a β-sheet structure described in the protein conformation classification database (SCOP/SCOP2) may be used, and a protein exhibiting a spectrum derived from the β-sheet structure by infrared spectroscopy analysis or circular dichroism analysis may be used. Further, the β-sheet portion may be increased in the presence of a crosslinking agent. The β-sheet structure may be amorphous or crystalline and may be crystallized. In FT-IR analysis using the ATR method, a protein for which the ratio of β-sheet structure per protein molecule is calculated to be 1% or more can be defined as a protein having a β-sheet structure. Specifically, it can be analyzed by the methods described in Nature Materials 2020, 19, 102-108. β-sheet structures are not only found within a single protein molecule, but are also formed by intermolecular association, as in fibroin. Since a β-sheet structure can be formed as long as it has an amino acid sequence to be a β-strand, the presence of more than 1% β-sheet structure per protein molecule is sufficient to adsorb crosslinking agents. The larger the proportion of the β-sheet structure, the easier the aromatic ring is to adsorb to the protein and the higher the crosslinking efficiency, which is preferable in terms of crosslinking.

The protein having a β-sheet structure is physically cross-linked by the crosslinking agent of this embodiment. A physical crosslink is one that results from a physical interaction between a protein and a crosslinking agent that does not involve a chemical covalent bond. The physical interactions include electrostatic interactions, van der Waals interactions, hydrogen bonds, and their combined interactions, which in this embodiment may be due to non-covalent adsorption interactions acting on the crosslinking agent and the β-sheet structural sites of the protein.

A preferred example of a protein having a β-sheet structure is fibroin. Fibroin used as a raw material is a protein molecule whose primary structure is a repeating region of a motif consisting of six amino acids (glycine-alanine-glycine-alanine-glycine-serine/tyrosine) and can be obtained by removing impurities from raw silk or cocoon produced by insects or arachnids. Examples of insects or arachnids include varieties described in Japanese Patent Laid-open No. 2018-150637.

### Protein Adhesive Agent

The present disclosure provides, as an embodiment, a protein adhesive agent comprising the above-mentioned crosslinking agent and a binder resin.

The binder resin is cured to strengthen cross-linking or to enhance adhesion between materials.

The binder resin may be dissolved in a solvent, dispersed as particles, cured by drying of the solvent, or cured by heating or irradiation with light. Further, the binder resin may function as a single body without containing solvent. The binder resin may be a resin or an unpolymerized resin. The resin may be a natural resin or a synthetic resin, and as the natural resin, for example, polyamine, cellulose, amylose, starch, collagen, chitin, keratin, and natural rubber may be used, and as the synthetic resin, for example, polyester resin, alkyd resin, urethane resin, epoxy resin, vinyl resin, acrylic resin, silicone resin, and fluororesin may be included. The content of the binder resin in the protein adhesive agent is not particularly specified, but preferably not less than 0.01 parts by weight and less than 50 parts by weight. The crosslinking agent of the present embodiment alone has a function of bonding proteins, but by containing more than 0.01 pts.wt. of the binder resin, the cross-linked proteins become more resistant to aggregation and destruction. In addition, from the viewpoint of handling and applicability, it is preferable not to have too much, and it is preferable to have less than 50 parts by weight.

### Protein Complex

The present disclosure provides, as an embodiment, a protein complex comprising the crosslinking agent and a protein having a β-sheet structure, wherein the protein having a β-sheet structure is crosslinked by a crosslinking agent for protein. The protein complex of the present embodiment has a β-sheet structure that is physically cross-linked by a crosslinking agent. In this embodiment, the protein having a β-sheet structure is preferably fibroin.

### Articles

The present disclosure provides, as an embodiment, an article in a solid form or in a gel form, comprising the protein complex described above. The article is used, for example, as a scaffolding material, a sensor device, a component of a sensor device, etc.

### Solid Form or Gel Form

The article of this embodiment has a solid form or a gel form. A solid form is tangible and includes, for example, a sheet-like, sponge-like, or fibrous form. The sheet-like article is a film-shaped solid, and the thickness is not specified. The sponge-like article is a porous solid and may be produced by, for example, volatilizing a solvent from a gel, although the method of production is not limited. The fibrous article is a fibrous solid and may be prepared, for example, by spinning from a solution, although the method of production is not limited.

The gel-like article has a three-dimensional network structure insoluble in a solvent or is a swollen body thereof. The solvent is preferably water from the viewpoint of stability of the protein complex but may be an organic solvent. The gel-like article may be an elastic body or a viscous body.

In articles in solid form or in gel form, the proteins contained therein may be uniformly dissolved, dispersed, or heterogeneous, and the proteins may form aggregates or fibrils. The article may also contain substances other than proteins in solid form or in gel form.

### Articles Wherein the First Protein and the Second Protein are Crosslinked by a Protein Crosslinking Agent

In addition, the present disclosure provides, as an embodiment, an article comprising a first layer comprising a first protein having a β-sheet structure, a second layer comprising a second protein having a β-sheet structure, and an adhesive layer between the first layer and the second layer, wherein the adhesive layer comprises a protein crosslinking agent, wherein the first protein and the second protein are crosslinked by the protein crosslinking agent.

### The First Protein and the Second Protein

The article of the present embodiment is formed by bonding a first layer comprising a first protein having a β-sheet structure and a second layer comprising a second protein having a β-sheet structure through an adhesive layer. The first protein and the second protein can be the same or different from each other as long as each of them has a β-sheet structure.

### Adhesive Layer

The article of this embodiment has an adhesive layer containing a protein crosslinking agent, and the adhesive layer may be a monolayer or thick. The first and second layers may each be either non-covalently bonded to the adhesive layer or interdiffused. The adhesive layer may include a binder resin.

### Compound

The present disclosure further provides, as an embodiment, a compound having a structural unit derived from one selected from the group consisting of polyethylene, polyvinyl alcohol, polyamines, polyamides, polyesters, and polyethers, and a group containing an aromatic ring, wherein the group containing an aromatic ring has at least one selected from a group consisting of a benzothiazole group, a benzoxazole group, a benzimidazole group, and a naphthylazo group, each of which may have a substituent.

The compound is preferably a polymer compound. The compound is not limited to use. The compound may be used as a protein crosslinking agent, a protein adhesive agent, or for other purposes. For example, the compound may be used as a liquid crystal material, a color former, a light emitting material, a light dimming material, a structural material, an ink, a toner, or as a dyeing agent, a dispersant, a surfactant, a coloring material, a filler, a surface modifier, and the like. The compound may be mixed with other substances or used alone.

### (Example)

Although the above embodiments will be described in more detail below with examples and comparative examples, the present disclosure is not limited by the following embodiments, unless the present disclosure goes beyond the scope thereof. As a side note, in terms of the amount of components, those described as “parts” and “%” are mass standards unless otherwise specified.

### Analytical Method

The analytical method used in the embodiment is as follows.

### (Determination of the Percentage Of Group Containing an Aromatic Ring in the Crosslinking Agent)

The percentage of group containing an aromatic ring was determined from UV-Vis absorption spectra of solutions of the crosslinking agents. A calibration curve was drawn by dissolving a compound containing an aromatic ring before addition to the crosslinking agent in water, the number of molecules of the monomer contained in the crosslinking agent was measured, and the value divided by the degree of polymerization of the polymer structure in the crosslinking agent was taken as the percentage of the group containing an aromatic ring.

(Molecular Weight Measurement by GPC)

Molecular weights were determined using an InfinityLab GPC/SEC system (manufactured by Agilent). The column used was Shim-pack Bio Diol-300, and the eluent was used wherein 0.1M phosphate buffer solution (pH7.4, manufactured by KISHIDA CHEMICAL Co., Ltd.) was supplemented with NaCl (manufactured by KISHIDA CHEMICAL Co., Ltd.) added to 0.2M.

### (Electrophoretic Determination of Fibroin Molecular Weight)

A microchip electrophoresis system, Agilent2100 Bioanalyzer Electrophoresis System (manufactured by Agilent), was used. The conditions are as follows. Microchips, separation matrices, fluorochromes, electrophoresis buffers, and molecular weight standard ladders were all from the Agilent Protein230 kit. Control samples were bovine serum albumin lyophilized powder, 96% (agarose gel electrophoresis) (manufactured by Sigma-Aldrich, molecular weight 66.5 kDa). Silk fibroin aqueous solution and control sample dilutions and concentrations: 8M urea aqueous solution were used to dilute the silk fibroin aqueous solution to 1.0 to 1.5 mass/vol % and the control sample to about 1.3 mass/vol %. The excitation wavelength was 630 nm, and the detection wavelength was 680 nm.

The molecular weight of silk fibroin was calculated using dedicated 2100 Expert software. The molecular weight of silk fibroin was calculated by the molecular weight calibration curve obtained from the data of the molecular weight standard ladder measured with the sample. As a side note, the electrophoretic band used for molecular weight calculation was the band with the most intense color.

### (Particle Size Measurement)

The sample was diluted with water to a scattering intensity of 1,000 to 10,000 cps and measured using a dynamic light scattering particle size measuring device (DLS, ELSZ-2000Z, manufactured by Otsuka Electronics Co., Ltd.). The median diameter of the volume reference was recorded.

### (Tensile Strength Measurement)

The sheet-shaped sample was cut into strips with a width of 10 mm and a length of 40 mm. After measuring the film thickness, tensile strength test was performed using a universal testing machine (Tensilon RTF-1250, manufactured by A & D Company, Limited). Tensile stress and elongation ratio with respect to the length of the specimen were measured when the specimen was fixed at 11.5 mm intervals in parallel fastening type jaw and fractured at different positions under 5 mm/min condition, and the tensile stress divided by the cross-sectional area was recorded as tensile strength.

### (Viscoelastic Measurement)

Viscoelasticity was measured using a rheometer (MCR³⁰², manufactured by Anton Paar). Slip was suppressed by attaching a #800 paper file to the tip of a 25 mm diameter parallel plate, and strain dispersion measurement of 0.001 to 0.01% strain was carried out under normal force of 0.5 N and frequency of 0.5 Hz condition, and storage modulus and loss modulus at 0.001% strain were recorded.

### (Measurement of Shear Adhesive Strength)

Tensile strength tests were performed on plate-like specimens using a universal material testing machine (Tensilon RTF-1250, manufactured by A & D Company, Limited). The specimens were fixed in parallel fastening jaws at 15 mm intervals and displaced at a rate of 5 mm/min. The tensile stress and the elongation ratio with respect to the specimen length at the time of fracture were measured, and the tensile stress divided by the adhesive area was recorded as the shear adhesive strength.

### Production of Compound Solutions

### (Example 1)

### Production of Compound 1 (Crosslinking Agent)

27 mg of Thioflavin T acid (manufactured by AAT Bioquest, Inc.) and 0.1 mL of N,N-dimethylformamide (hereinafter abbreviated as “DMF”) were dissolved in 1 mL of dichloromethane (hereinafter abbreviated as “DCM”) and stirred under ice. 0.15 mL of oxalyl chloride was dissolved in 1 mL of DCM. Oxalyl chloride solution was added dropwise to Thioflavin T acid solution. After stirring overnight, the solvent was distilled off under reduced pressure and dissolved again in 1 mL of DMF to obtain a Thioflavin T acid acid chloride solution. A solution of PVA, in which 12 mg of polyvinyl alcohol (manufactured by Sigma-Aldrich, Mw13,000-23,000, 87 to 89% hydrolyzed, hereinafter abbreviated as “PVA”) was dissolved in 1 mL of DMF, was mixed with a solution of Thioflavin T acid acid chloride, and the mixture was stirred at 120° C. under nitrogen for 6 hours. The solvent was evaporated under reduced pressure and redissolved in 2 mL of pure water. Gel filtration was performed using a PD-10 column (manufactured by Cytiva) to extract the high molecular weight components to give 2.9 g of a solution of compound 1. The reaction flow is shown below.

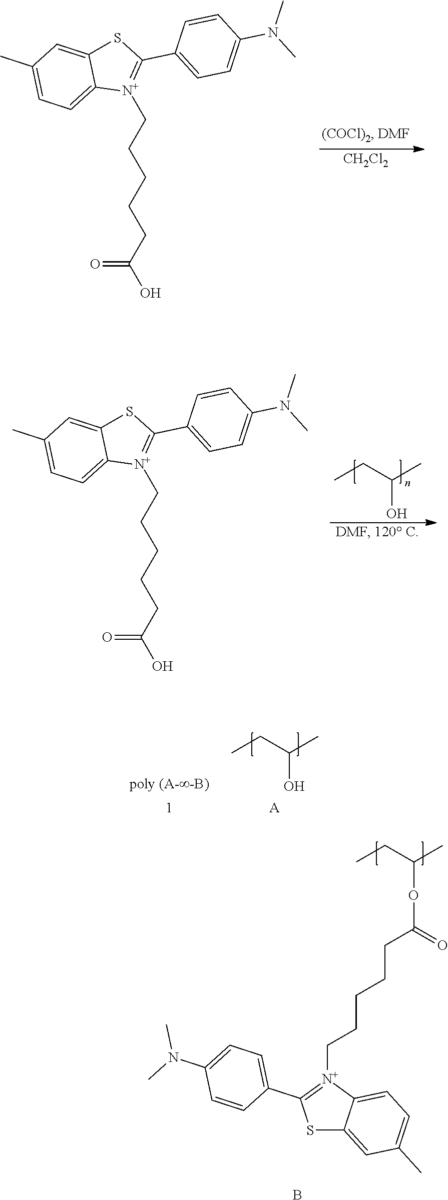

The obtained compound was confirmed to be the target compound by molecular weight analysis using GPC. As a result of measuring the absorbance, the percentage of groups containing aromatic rings in the crosslinking agent was 10%.

### (Example 2)

### Production of Compound 2 (Compound Containing an Aromatic Ring) and Compound 3 (Crosslinking Agent)

To 15 mL of N, N-dimethylformamide were added 1.10 g of 2-(4-aminophenyl)benzothiazole (manufactured by Tokyo Chemical Industry Co., Ltd.) and 0.40 g of 3-bromopropionic acid (manufactured by Tokyo Chemical Industry Co., Ltd.), and the mixture was refluxed under a nitrogen atmosphere for 4 hours. After being brought to room temperature and the solvent was distilled off, it was purified by silica gel column chromatography to obtain 201 mg (yield 26%) of a white solid of compound 2. Identification was carried out by 1H-NMR (manufactured by Bruker, Bruker Avance NEO 500, Resonance Frequency: 500 MHZ) measurement equipment. 1H-NMR (DMSO-d6) (ppm): 12.30 (br, 1H), 8.06 (d, 1 H, J=7.8 Hz), 7.93 (d, 1 H, J=7.8 Hz), 7.86 (d, 2 H, J=9.0 Hz), 7.51-7.48 (m, 1H), 7.40-7.37 (m, 1H), 6.74 (d, 2 H, J=9.0 Hz), 6.53 (t, 1 H, J=5.5 Hz), 3.35 (m, 2H), 2.58 (m, 2H).

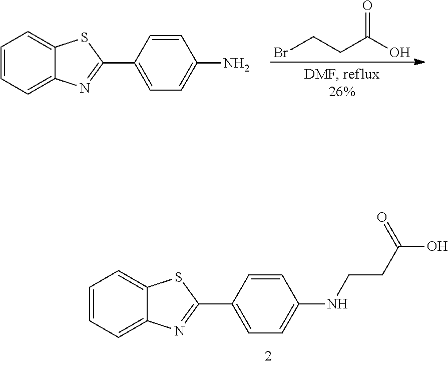 [SMILES: Nc1ccc(-c2nc3ccccc3s2)cc1 ; O=C(O)CCBr ; O=C(O)CCNc1ccc(-c2nc3ccccc3s2)cc1]

27 mg of the white solid of the obtained compound 2 and 0.1 mL of N, N-dimethylformamide (hereinafter abbreviated as “DMF”) were dissolved in 1 mL of dichloromethane (hereinafter abbreviated as “DCM”), and the mixture was stirred under ice cooling. 0.15 mL of oxalyl chloride was dissolved in 1 mL of DCM. The oxalyl chloride solution was added dropwise to Thioflavin T acid solution. After stirring overnight, the solvent was distilled off under reduced pressure and dissolved again in 1 mL of DMF to obtain a Thioflavin T acid acid chloride solution. A solution of PVA in which 12 mg of polyvinyl alcohol (manufactured by Sigma-Aldrich, Mw13,000-23,000, 87 to 89% hydrolyzed, hereinafter abbreviated as “PVA”) was dissolved in 1 mL of DMF was mixed with the solution of Thioflavin T acid acid chloride, and the mixture was stirred at 120° C. under nitrogen for 6 hours. The solvent was evaporated under reduced pressure and redissolved in 2 mL of pure water. Gel filtration was performed using a PD-10 column (manufactured by Cytiva) to extract the high molecular weight components to give 2.9 g of a solution of compound 3. The reaction flow is shown below.

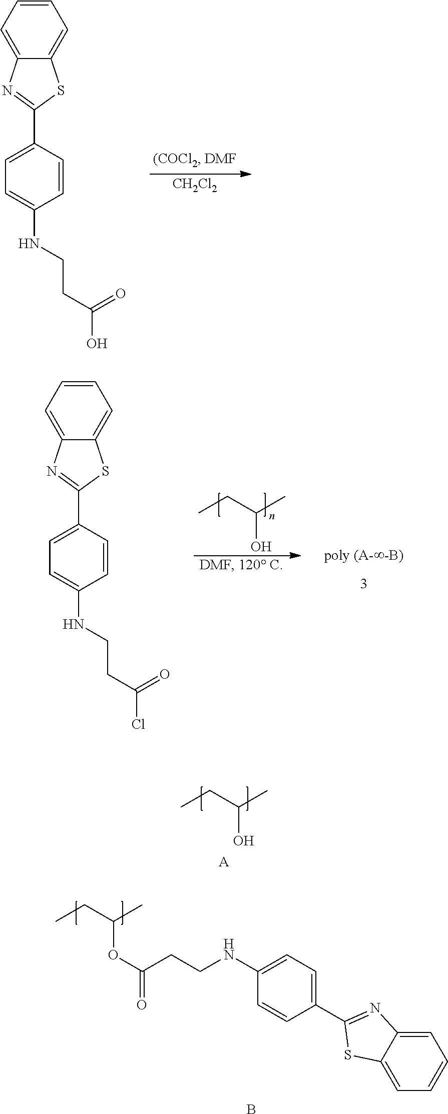

The obtained compound was confirmed to be the target compound by molecular weight analysis using GPC. As a result of measuring the absorbance, the percentage of groups containing aromatic rings in the crosslinking agent was 9%.

### (Example 3)

### Production of Compound 4 (Compound Containing an Aromatic Ring) and Compound 5 (Crosslinking Agent)

To 20 mL of tetrahydrofuran were added 1.01 g of 2-(2-hydroxyphenyl) benzothiazole (manufactured by Tokyo Chemical Industry Co., Ltd.), 0.67 g of 3-bromopropionic acid (manufactured by Tokyo Chemical Industry Co., Ltd.), and 1.52 g of potassium carbonate (manufactured by KISHIIDA CHEMICAL CO., LTD.), and refluxed under a nitrogen atmosphere for 3 hours. After returning to room temperature, 10 mL of methanol was added. The precipitate obtained by filtration was dried to give 57 mg (4% yield) of a white solid of compound 4. Identification was carried out by 1H-NMR (manufactured by Bruker, Bruker Avance NEO 500, Resonance Frequency: 500 MHz) measurement equipment. 1H-NMR (DMSO-d6) (ppm): 12.52 (br, 1H), 8.50-8.48 (m, 1H), 8.13 (d, 1 H, J=8.0 Hz), 8.10 (d, 1 H, J=8.0 Hz), 7.61-7.56 (m, 2H), 7.50-7.46 (m, 1H), 7.37-7.36 (m, 1H), 7.23-7.20 (m, 1H), 4.52 (t, 2 H, J-6.0 Hz), 2.99 (t, 2 H, J-6.0 Hz).

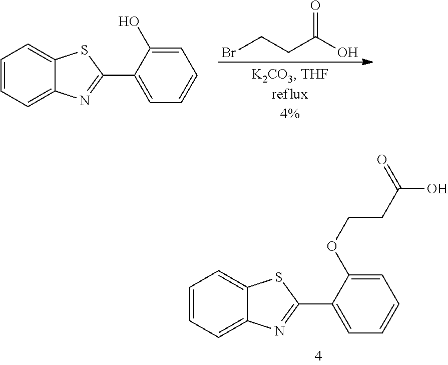

27 mg of the white solid of the obtained compound 4 and 0.1 mL of N, N-dimethylformamide (hereinafter abbreviated as “DMF”) were dissolved in 1 mL of dichloromethane (hereinafter abbreviated as “DCM”), and the mixture was stirred under ice cooling. 0.15 mL of oxalyl chloride was dissolved in 1 mL of DCM. The oxalyl chloride solution was added dropwise to Thioflavin T acid solution. After stirring overnight, the solvent was evaporated under reduced pressure and dissolved again in 1 mL of DMF to obtain a Thioflavin T acid acid chloride solution. A solution of PVA in which 12 mg of polyvinyl alcohol (manufactured by Sigma-Aldrich, Mw13,000-23,000, 87 to 89% hydrolyzed, hereinafter abbreviated as “PVA”) was dissolved in 1 mL of DMF was mixed with the solution of Thioflavin T acid acid chloride, and the mixture was stirred at 120° C. under nitrogen for 6 hours. The solvent was evaporated under reduced pressure and redissolved in 2 mL of pure water. A PD-10 column (manufactured by Cytiva) was used for gel filtration to extract the high molecular weight components to give 2.9 g of a solution of compound 5. The reaction flow is shown below.

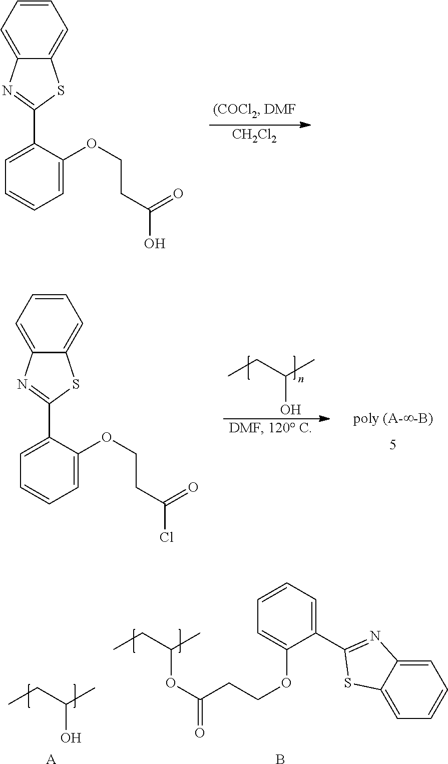

The obtained compound was confirmed to be the target compound by molecular weight analysis using GPC. As a result of measuring the absorbance, the percentage of groups containing aromatic rings in the crosslinking agent was 12%.

### Preparation of Fibroin Aqueous Solution

### (Silk Scouring)

In a 5 L glass beaker, 4.5 L of ultrapure water was heated and boiled, then 8.48 g of sodium carbonate (manufactured by KISHIDA CHEMCAL Co., Ltd.) was added to make a 0.02 mol/L sodium carbonate solution, and 10 g of cut cocoons of domestic silkworms (manufactured by Tajima Trading Co., Ltd.) cut into 1 cm cubes were added and heated for 30 minutes to obtain silk from which sericin was removed. The silk was washed with cold ultrapure water, drained, and dried in a draft overnight to obtain the silk after scouring.

### (Preparation of Fibroin Aqueous Solution)

To a graduated cylinder, 0.86 g of lithium bromide (manufactured by KISHIDA CHEMICAL Co., Ltd.) was added, and the volume was increased to 10 mL to obtain a lithium bromide solution of 9.3 mol/L. A 100 mL glass beaker was filled with 3.0 g of silk after scouring, and 14.8 mL of the 9.3 mol/L lithium bromide solution was added so that the silk after scouring was completely immersed, and then dissolved in an oven at 60° C. for 2 hours to obtain a clear aqueous solution.

### (Purification of Aqueous Fibroin Solution)

Dialysis was performed by injecting 19 mL of the obtained clear aqueous solution into a dialysis cassette (manufactured by Thermo Scientific) with a fractionated molecular weight of 3500 fractions and a capacity of 30 mL by syringe and immersing in 2 L of ultrapure water. Water was changed once every 8 hours for a total of 48 hours of dialysis to remove small molecule contaminants and lithium ions. The obtained aqueous solution was rotated in a centrifuge CR⁷N (manufactured by Yamato Scientific Co., Ltd.) at 11000 rpm/4° C. for 20 minutes to remove impurities, and an aqueous solution of fibroin was obtained. The molecular mass determined by electrophoresis was 100 kDa. The obtained fibroin solution was analyzed by DLS, and the median diameter was 16 nm.

### (Evaluation of Adsorptivity of Groups Containing Aromatic Rings to Proteins Having a β-Sheet Structure)

Methanol solutions (0.02 mM, 0.15 mL) of Thioflavin T acid, compound 2, compound 4, and 7-(diethylamino) coumarin-3 carboxylic acid, which are compounds having a group containing an aromatic ring, were prepared and added to 10 mg of silk fibroin powder (manufactured by Sigma-Aldrich). After distillation of methanol by air-drying, 0.15 mL of water was added. After stirring for 5 minutes, the supernatant was collected. The concentration of groups containing aromatic rings in the collected supernatant liquid and the percentage of adsorption of groups containing aromatic rings to fibroin were calculated by absorbance measurement, and the adsorptivity of groups containing aromatic rings to proteins having a β-sheet structure was evaluated by the following criteria.

“Evaluation A” and “Evaluation B” indicate the level at which groups containing aromatic rings adsorb strongly to proteins having a β-sheet structure, and “Evaluation C” indicates the low adsorptivity of groups containing aromatic rings.

### (Criteria)

A: 90% or more
B: 60% or more but less than 90%
C: Less than 60%

Table 1 shows the adsorption rate of compounds having groups containing aromatic rings on fibroin and the results of the evaluation of the adsorptivity of groups containing aromatic rings.

TABLE 1

| Compound having group | | | | 7- (Diethylamino) Coumarin -3 |
|---|---|---|---|---|
| containing aromatic ring | Thioflavin T acid | Compound 2 | Compound 4 | Carboxylic Acid |
| Adsorption rate to | 91 | 79 | 71 | 55 |
| fibroin (%) | | | | |
| Adsorptivity | A | B | B | C |

### Production of Protein Complexes

### (Example 4)

10 mL of a solution of compound 1 was mixed and stirred with 10 mL of a 40 mg/mL aqueous solution of fibroin to give a protein complex solution 1. When the particle size was confirmed using DLS, the median diameter increased to 28 nm, and it was confirmed that the fibroin was crosslinked to form a complex.

### (Example 5)

10 mL of a solution of compound 3 was mixed and stirred with 10 mL of a 40 mg/mL aqueous solution of fibroin to give a protein complex solution 2. When the particle size was confirmed using DLS, the median diameter increased to 29 nm, and it was confirmed that the fibroin was crosslinked to form a complex.

### (Example 6)

10 mL of a solution of compound 5 was mixed and stirred with 10 mL of a 40 mg/mL aqueous solution of fibroin to give a protein complex solution 3. When the particle size was confirmed using DLS, the median diameter increased to 27 nm, and it was confirmed that the fibroin was crosslinked to form a complex.

### (Comparative Example 1)

Instead of a solution of compound 1, 10 mL of a 1 mg/mL aqueous solution of LPVA was mixed and stirred with 10 mL of a 40 mg/mL aqueous solution of fibroin to obtain a protein complex solution 4. When the particle size was confirmed using DLS, the median diameter was 16 nm, and it was confirmed that no complex was formed.

The physical properties of the protein complexes of Example 4 to 6 and Comparative Example 1 are shown in Table 2.

TABLE 2

| | | | | Median |
|---|---|---|---|---|
| | | | | diameter |
| | Compound | Ptotein | Shape | [nm] |
| Example 4 | Compound 1 | Silk fibroin | Complex 1 | 28 |
| Example 5 | Compound 3 | Silk fibroin | Complex 2 | 29 |
| Example 6 | Compound 5 | Silk fibroin | Complex 3 | 27 |
| Comparative | PVA | Silk fibroin | Complex 4 | 16 |
| Example 1 | | | | |

### Production of Article in Sheet Form

### (Example 7)

10 mL of the protein complex solution 1 was taken, poured into a vat having a bottom area of 80 cm2, dried in an oven at 40° C. for 24 hours, and the obtained sheet was immersed in methanol for 2 minutes to allow the crystallization of fibroin to proceed, and dried in a draft for 30 minutes to obtain the sheet body 1. Tensile strength test was performed on the obtained sheet body 1, and tensile strength was 78 MPa and elongation was 6%.

### (Example 8)

10 mL of the protein complex solution 2 was taken, poured into a vat having a bottom area of 80 cm2, dried in an oven at 40° C. for 24 hours, and the obtained sheet was immersed in methanol for 2 minutes to allow the crystallization of fibroin to proceed, and dried in a draft for 30 minutes to obtain the sheet body 2. Tensile strength test was performed on the obtained sheet body 2, and tensile strength was 78 MPa and elongation was 7%.

### (Example 9)

10 mL of the protein complex solution 3 was taken, poured into a vat having a bottom area of 80 cm2, dried in an oven at 40° C. for 24 hours, and the obtained sheet was immersed in methanol for 2 minutes to allow the crystallization of fibroin to proceed, and dried in a draft for 30 minutes to obtain the sheet body 3. Tensile strength test was performed on the obtained sheet body 3, and tensile strength was 78 MPa and elongation was 8%.

### (Comparative Example 2)

10 mL of the protein complex solution 4 was taken, poured into a vat having a bottom area of 80 cm², dried in an oven at 40° C. for 24 hours, and the obtained sheet was immersed in methanol for 2 minutes to allow the crystallization of fibroin to proceed, and dried in a draft for 30 minutes to obtain the sheet body 4. Tensile strength test was performed on the obtained sheet body 4, and tensile strength was 42 MPa and elongation was 5%.

### (Comparative Example 3)

10 mL portion of a 40 mg/mL aqueous solution of fibroin was taken, poured into a vat having a bottom area of 80 cm2, dried in an oven at 40° C. for 24 hours, and the obtained sheet was immersed in methanol for 2 minutes to allow the crystallization of fibroin to proceed, and dried in a draft for 30 minutes to obtain the sheet body 5. Tensile strength test was performed on the obtained sheet body 5, and tensile strength was 60 MPa and elongation was 5%.

The physical properties of Example 7 to 9, Comparative Example 2, and Comparative Example 3 are shown in Table 3.

TABLE 3

| | | | | Tensile | |
|---|---|---|---|---|---|
| | | | | strength | Elongation |
| | Compound | Protein | Shape | [MPa] | [%] |
| Example 7 | Compound 1 | Silk fibroin | Sheet Body 1 | 78 | 6 |
| Example 8 | Compound 3 | Silk fibroin | Sheet Body 2 | 78 | 7 |
| Example 9 | Compound 5 | Silk fibroin | Sheet Body 3 | 78 | 8 |
| Comparative | PVA | Silk fibroin | Sheet Body 4 | 42 | 5 |
| Example 2 | | | | | |
| Comparative | None | Silk fibroin | Sheet Body 5 | 60 | 5 |
| Example 3 | | | | | |

### Production of Protein Sheet Body 2

### (Example 10)

10 mg of compound 1 powder and 100 mg of fibroin powder (manufactured by Sigma-Aldrich) were added to 10 mL of hexafluoro-2 propanol, stirred at room temperature using a mix rotor, and filtered through a filter (Millex-AP, manufactured by Millipore) to obtain a mixed solution. Using 1 mL of the mixed solution, a sheet of fibroin fibers was prepared and collected on an aluminum dish using an electrospinning apparatus (ANON-03, manufactured by MECC). After methanol was added to the collected sheet, methanol was removed and dried overnight to obtain a sheet body composed of fibroin fibers. The measurement of the weight of the fibroin sheet cut into 20 mm×40 mm size confirmed that it was 1.25 μg/mm² fibroin sheet.

### Production of Article in Gel Form

### (Example 11)

6 mL of the protein complex solution 1 was treated with an ultrasonic homogenizer (UD-201, manufactured by TOMY SEIKO Co., Ltd.) at a strength of 6 for 5 minutes, poured into a petri dish with a bottom area of 12.5 cm², sealed, and allowed to stand for 2 weeks to obtain the gel body. When viscoelastic measurement was performed on the obtained gel body 1, and the storage modulus was 4×10⁴ GPa and the loss modulus was 3×10³ GPa.

### (Example 12)

6 mL of the protein complex solution 2 was treated with an ultrasonic homogenizer (UD-201, manufactured by TOMY SEIKO Co., Ltd.) at a strength of 6 for 5 minutes, poured into a petri dish with a bottom area of 12.5 cm², sealed, and allowed to stand for 2 weeks to obtain the gel body. When viscoelastic measurement was performed on the obtained gel body 2, the storage modulus was 5×10⁴ GPa and the loss modulus was 3×10³ GPa.

### (Example 13)

6 mL of the protein complex solution 3 was treated with an ultrasonic homogenizer (UD-201, manufactured by TOMY SEIKO Co., Ltd.) at a strength of 6 for 5 minutes, poured into a petri dish with a bottom area of 12.5 cm², sealed, and allowed to stand for 2 weeks to obtain the gel body. When viscoelastic measurement was performed on the obtained gel body 3, and the storage modulus was 6×10⁴ GPa and the loss modulus was 3×10³ GPa.

### (Comparative Example 4)

6 mL of the protein complex solution 4 was treated with an ultrasonic homogenizer (UD-201, manufactured by TOMY SEIKO Co., Ltd.) at a strength of 6 for 5 minutes, poured into a petri dish with a bottom area of 12.5 cm², sealed, and allowed to stand for 2 weeks to obtain the gel body. When viscoelastic measurement was performed on the obtained gel body 4, the storage modulus was 2×10⁴ GPa and the loss modulus was 2×10³ GPa.

### (Comparative Example 5)

6 mL of a 40 mg/mL aqueous solution of fibroin was treated with an ultrasonic homogenizer (UD-201, manufactured by TOMY SEIKO Co., Ltd.) at a strength of 6 for 5 minutes, poured into a petri dish with a bottom area of 12.5 cm², sealed, and allowed to stand for 2 weeks to obtain the gel body. When viscoelastic measurement was performed on the obtained gel body 5, and the storage modulus was 3×10⁴ GPa and the loss modulus was 3×10³ GPa.

The physical properties of Example 11 to 13, Comparative Example 4, and Comparative Example 5 are shown in Table 4.

TABLE 4

| | | | | Storage | Loss |
|---|---|---|---|---|---|
| | | | | modulus | modulus |
| | Compound | Protein | Shape | [GPa] | [GPa] |
| Example 11 | Compound 1 | Silk fibroin | Gel Body 1 | 4 × 10⁴ | 3 × 10³ |
| Example 12 | Compound 3 | Silk fibroin | Gel Body 2 | 5 × 10⁴ | 3 × 10³ |
| Example 13 | Compound 5 | Silk fibroin | Gel Body 3 | 6 × 10⁴ | 3 × 10³ |
| Comparative | PVA | Silk fibroin | Gel Body 4 | 2 × 10⁴ | 2 × 10³ |
| Example 4 | | | | | |
| Comparative | None | Silk fibroin | Gel Body 5 | 3 × 10⁴ | 3 × 10³ |
| Example 5 | | | | | |

### Production of Article in Sponge Form

### (Example 14)

Sodium chloride (Particle size 500-710 μm, manufactured by Tokyo Chemical

Industry Co., Ltd.) was added to a 2.5 ml syringe, and 0.5 mL of protein complex solution 1 was added. The syringe was extruded so that the tip of the piston was in contact with sodium chloride. After incubation in a 37° C. oven for 24 hours, the syringe was broken to obtain the gel body. After that, it was washed three times with 2 L of Milli-Q water and freeze-dried to obtain an article in a sponge form. It was confirmed that the hole of about 500 μm was opened when it was cut and observed by electron microscope.

### Production of article (adhesive layer structure) comprising an adhesive layer and two protein layers

### (Example 15)

The PET film (Lemiller 188 S10, manufactured by Toray Industries, Inc.) was cut into 2 strips with a width of 10 mm and a length of 40 mm, and a protein solution 1 was applied to each strip using a bar coater so that the wet film thickness was 10 μm, dried for 24 hours, and immersed in methanol for 2 minutes, and the methanol was dried off and dried overnight. 10 μL of the compound 1 was dropped on the solution-coated surface of one film, and bonded to the solution-coated surface of the other film to obtain a 70 mm long protein adhesive layer structure 1 (article 1) having an adhesive surface of 10 mm×10 mm. After drying for 24 hours with the adhesive surface sandwiched between the clips, the shear bond strength test was carried out. As a result, the shear bond strength was 0.40 MPa and the elongation was 0.6%.

### (Example 16)

The PET film (Lemiller 188 S10, manufactured by Toray Industries, Inc.) was cut into 2 strips with a width of 10 mm and a length of 40 mm, and a protein solution 2 was applied to each strip using a bar coater so that the wet film thickness was 10 μm, dried for 24 hours, and immersed in methanol for 2 minutes, and the methanol was dried off and dried overnight. 10 μL of the compound 3 was dropped on the solution-coated surface of one film, and bonded to the solution-coated surface of the other film to obtain a 70 mm long protein adhesive layer structure 2 (article 2) having an adhesive surface of 10 mm×10 mm. After drying for 24 hours with the adhesive surface sandwiched between the clips, the shear bond strength test was carried out. As a result, the shear bond strength was 0.50 MPa and the elongation was 0.5%.

### (Example 17)

The PET film (Lemiller 188 S10, manufactured by Toray Industries, Inc.) was cut into 2 strips having a width of 10 mm and a length of 40 mm, and a protein solution 3 was applied to each strip using a bar coater so that the wet film thickness was 10 μm, dried for 24 hours, and immersed in methanol for 2 minutes, and the methanol was dried off and dried overnight. 10 μL of the compound 5 was dropped on the solution-coated surface of one film, and bonded to the solution-coated surface of the other film to obtain a 70 mm long protein adhesive layer structure 3 (article 3) having an adhesive surface of 10 mm×10 mm. After drying for 24 hours with the adhesive surface sandwiched between the clips, the shear bond strength test was carried out. As a result, the shear bond strength was 0.60 MPa and the elongation was 0.5%.

### (Comparative Example 6)

Two sheets of PET films (Lumirr 188 S10, manufactured by Toray Industries, Inc.) were cut into strip shapes having a width of 10 mm and a length of 40 mm, and a protein complex solution 4 was applied to each of them and dried, and then 10 μL of a 1 mg/mLPVA aqueous solution was dropped onto the solution applied surface of one film and bonded to the other solution applied surface to obtain a protein adhesive layer structure 4 (article 4) having a length of 70 mm and an adhesive surface of 10 mm×10 mm. After drying for 24 hours with the adhesive surface sandwiched between the clips, the shear bond strength test was carried out. As a result, the shear bond strength was 0.03 MPa and the elongation was 0.1%.

The physical properties of the article (adhesive layer structure) of Example 15 to 17, and Comparative Example 6 are shown in Table 5.

TABLE 5

| | | | | shear bond strength | Elongation |
|---|---|---|---|---|---|
| | Compound | Protein | Shape | [MPa] | [%] |
| Example 15 | Compound 1 | Silk fibroin | Adhesive | 0.40 | 0.6% |
| | | | Structure 1 | | |
| Example 16 | Compound 3 | Silk fibroin | Adhesive | 0.50 | 0.5% |
| | | | Structure 2 | | |
| Example 17 | Compound 5 | Silk fibroin | Adhesive | 0.60 | 0.5% |
| | | | Structure 3 | | |
| Comparative | PVA | Silk fibroin | Adhesive | 0.03 | 0.1% |
| Example 6 | | | Structure 4 | | |

According to the present disclosure, a protein having a β-sheet structure can be crosslinked by a simple operation, and a crosslinked protein complex and protein structure can be provided.

Further, according to the crosslinking agent of the present disclosure, proteins can be crosslinked without heat or chemical reaction, and the crosslinking agent has the advantage that can be applied to proteins fragile by heat or chemical reaction.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A protein crosslinking agent for a protein having a β-sheet structure, comprising:
a structural unit derived from one selected from the group consisting of polyethylene, polyvinyl alcohol, polyamine, polyamide, polyester, and polyether, and
a group containing an aromatic ring,
wherein the group containing an aromatic ring has at least one selected from the group consisting of a benzothiazole group, a benzoxazole group, a benzimidazole group, and a naphthylazo group, each of which may have a substituent.

2. The crosslinking agent of claim 1, wherein the group containing an aromatic ring comprises at least one of a benzothiazole group and a naphthylazo group.

3. The crosslinking agent of claim 1, wherein the group containing an aromatic ring is any one of monovalent groups represented by formula (1-1), formula (2-1), formula (3-1), formula (4), formula (5-1), or formula (9-1) 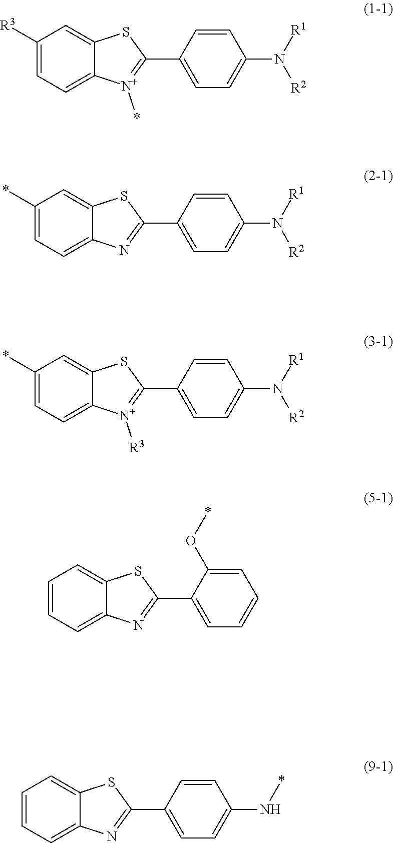
in the above formula (1-1), formula (2-1), formula (3-1), formula (5-1), and formula (9-1),
R¹ to R³ independently represents a hydrogen atom, a substituted or unsubstituted aliphatic hydrocarbon group having 1 to 4 carbon atoms, a substituted or unsubstituted aryl or aralkyl group having 6 to 10 carbon atoms, or an alkoxy group, and
symbol * indicates a bonding site 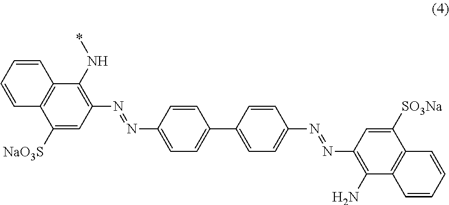
in the above formula (4), symbol * indicates a bonding site.

4. A protein adhesive agent comprising a crosslinking agent according to claim 1, and a binder resin.

5. A protein complex comprising a crosslinking agent according to claim 1 and a protein having a β-sheet structure,
wherein the protein having a β-sheet structure is crosslinked by the protein crosslinking agent.

6. The protein complex of claim 5, wherein the protein having a β-sheet structure is a fibroin.

7. An article in a solid form or in a gel form, comprising a protein complex according to claim 5.

8. The article of claim 7, wherein the solid form is either a sheet-like form, a sponge-like form, or a fibrous form.

9. An article comprising:
a first layer comprising a first protein having a β-sheet structure,
a second layer comprising a second protein having a β-sheet structure, and
an adhesive layer between the first layer and the second layer,
wherein the adhesive layer comprises a protein crosslinking agent according to claim 1,
wherein the first protein and the second protein are crosslinked by the protein crosslinking agent.

10. The article of claim 9, wherein the adhesive layer comprises a binder resin.

11. A compound comprising a structural unit derived from one selected the group consisting of polyethylene, polyvinyl alcohol, polyamine, polyamide, polyester, and polyether, and
a group containing an aromatic ring,
wherein the group containing an aromatic ring has at least one selected from the group consisting of a benzothiazole group, a benzoxazole group, a benzimidazole group, and a naphthylazo group, each of which may have a substituent.

12. The compound of claim 11, wherein the group containing an aromatic ring comprises at least one of a benzimidazole group and a naphthylazo group.

13. The compound of claim 11 wherein the group containing an aromatic ring is any one of the monovalent groups represented by formula (1-1), formula (2-1), formula (3-1), formula (4), formula (5-1), or formula (9-1) 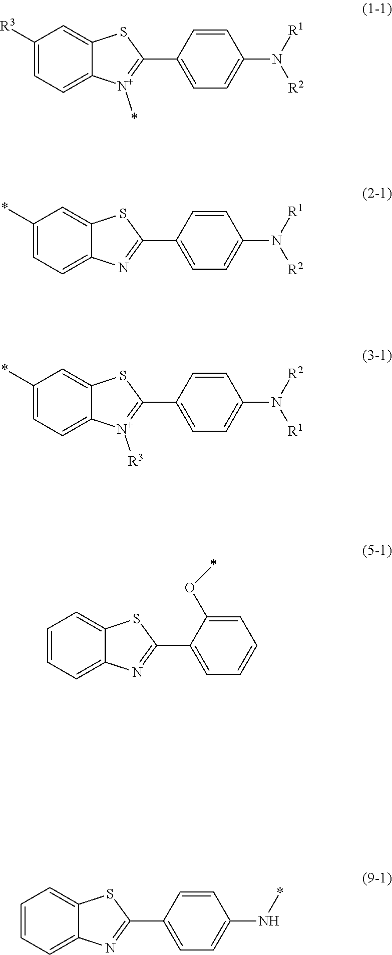
in the above formula (1-1), formula (2-1), formula (3-1), formula (5-1), and formula (9-1),
R¹ to R³ independently represent hydrogen atoms, substituted or unsubstituted aliphatic hydrocarbon groups having 1 to 4 carbon atoms, substituted or unsubstituted aryl or aralkyl groups having 6 to 10 carbon atoms, or alkoxy groups, and
symbol * indicates a bonding site 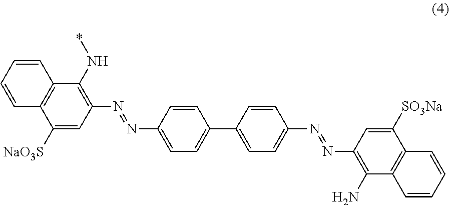
in the above formula (4), symbol * indicates a bonding site.

14. A method for crosslinking a protein comprising a step of mixing a protein crosslinking agent according to claim 1 with the protein.

15. A method for adhering proteins comprising a step of mixing a protein adhesive agent according to claim 4 and the proteins.

16. A method for manufacturing an article comprising:
a step of applying a protein crosslinking agent according to claim 1 to at least one of a first sheet comprising a first protein having a β-sheet structure and a second sheet comprising a second protein having a β-sheet structure; and
a step of sticking the first sheet comprising a first protein having a β-sheet structure and the second sheet comprising a second protein having a β-sheet structure via the protein crosslinking agent.

17. A method for manufacturing an article comprising:
a step of applying a protein adhesive agent according to claim 4 to at least one of a first sheet comprising a first protein having a β-sheet structure and a second sheet comprising a second protein having a β-sheet structure; and
a step of sticking the first sheet comprising a first protein having a β-sheet structure and the second sheet comprising a second protein having a β-sheet structure via the protein adhesive agent.
